(19) United States
(12) Patent Application Publication
(10) Pub. No.: US 2024/0237919 A1
(43) Pub. Date: Jul. 18, 2024

(54) VIBRATION SENSOR AND DEVICE FOR MEASURING PERIODIC VITAL SIGNALS EMITTED BY THE HUMAN OR ANIMAL BODY

(71) Applicant: Wormsensing, Grenoble Cedex 9 (FR)

(72) Inventors: Thomas Lorne, Eguilles (FR); Jude Guelfucci, Grenoble (FR); Ismail Degirmencioglu, Nantua (FR); Lamine Benaissa, Grenoble (FR)

(21) Appl. No.: 18/561,901

(22) PCT Filed: May 11, 2022
(86) PCT No.: PCT/FR2022/050902

§ 371 Date: Nov. 17, 2023

(30) Foreign Application Priority Data
May 18, 2021 (FR) FR2105201

(51) Int. Cl. A61B 5/11; A61B 5/00; A61B 5/024

(52) U.S. Cl. CPC A61B 5/11; A61B 5/02438; A61B 5/6832; A61B 2562/166

(57) ABSTRACT

A vibration sensor for measuring a vital periodic signal from an individual comprises: —a stack of layers including an active layer made of piezoelectric material and two contact electrodes arranged on the active layer, the active layer having a thickness less than or equal to 20 microns and a Young's modulus greater than or equal to 60 GPa; —a flexible support layer including a printed circuit comprising two electrical terminals; and—an electrical connection layer arranged between the stack and the support layer for connecting each electrode to a terminal.

### CROSS REFERENCE TO RELATED APPLICATIONS

This application is a national phase entry under 35 U.S.C. § 371 of International Patent Application PCT/FR2022/050902, filed May 11, 2022, designating the United States of America and published as International Patent Publication WO 2022/243624 A1 on Nov. 24, 2022, which claims the benefit under Article 8 of the Patent Cooperation Treaty to French Patent Application Serial No. FR2105201, filed May 18, 2021.

### TECHNICAL FIELD

The present disclosure relates to the field of collecting periodic vital signals emitted by the human/animal body, signals that include, in particular, heart beats and respiratory rate. It relates to, in particular, a vibration sensor and a device equipped with the sensor, allowing the measurement of the periodic vital signals, by being applied against the body, in contact with the skin or with an intermediate layer (clothing, fur, leather, etc.), in calm or noisy environments.

### BACKGROUND

Phonocardiography is a non-invasive acoustic method for measuring heart rate. From the phonocardiograms (PCG) obtained with this method, it is possible, after analysis, to deduce different indicators indicating the general operation of the heart and/or the state of the subject (health, stress, emotions, fatigue, etc.). For example, heart rate variability (HRV) is a particularly relevant indicator frequently used to determine physiological responses related to emotions, stress, fatigue or sleep.

Although proven and recognized by the scientific community, phonocardiography nevertheless has certain constraints that today limit its widespread adoption. Indeed, current devices require, for the most part, intimate contact with the subject (directly on the skin), knowledge of the appropriate positioning of the measuring device and of the pressure to be exerted on the skin, and a calm sound environment. This is, in particular, the case of the portable device described in document KR101957110, which comprises a piezoelectric ceramic sensor, intended to be directly in contact with the user's skin, and an adhesive ring making it possible to maintain contact with the skin.

### BRIEF SUMMARY

The present disclosure aims to remedy all or some of the aforementioned drawbacks. It relates to, in particular, a compact device, able to capture and analyze the periodic vital signals, by being applied against the body of an individual, in contact with the skin or with an intermediate layer (clothing, fur, leather, etc.), and does so in calm or noisy environments, whether the subject is immobile or moving.

The present disclosure relates to a vibration sensor for measuring at least one periodic vital signal of an individual, comprising:
a stack of layers extending parallel to a main plane and including an active layer made of piezoelectric material and two contact electrodes arranged on at least one face of the active layer, the active layer having a thickness less than or equal to 20 microns and a Young's modulus greater than or equal to 60 GPa,
a flexible support layer configured to transmit a deformation to the active layer of the stack of layers at each pulse of the vital signal, the support layer (30) extending parallel to the main plane and including a printed circuit comprising two electrical terminals, and
an electrical connection layer, arranged between the stack of layers and the support layer, to connect each contact electrode to an electrical terminal, the vibration sensor being intended to be in contact with the individual, on the side of the support layer.

According to other advantageous non-limiting features of the present disclosure, taken alone or according to any technically feasible combination:
the vibration sensor comprises an impedance matching layer, having an acoustic impedance between 5×10⁵ Pa*s/m and 3×10⁶ Pa*s/m, and arranged on a face of the support layer opposite the one in contact with the electrical connection layer;
the piezoelectric material of the active layer is chosen from ceramics in monocrystalline, poly-crystalline or composite form;
the contact electrodes have a cumulative thickness of less than twice the thickness of the active layer;
the support layer is self-supporting and has a thickness of less than or equal to 500 microns;
the impedance matching layer has a thickness greater than or equal to 10 microns;
the electrical connection layer is formed by an interposer or by an anisotropic conductive film;
the support layer includes a membrane disposed on a face of the printed circuit opposite the one in contact with the electrical connection layer;
the stack of layers and the support layer, respectively, have a first surface area and a second area, in the main plane, the first surface area being less than or equal to 30% of the second surface;
the support layer comprises a stiffening structure, rigidly connected to a peripheral zone of the support layer;
the printed circuit comprises a wire connection element, for connecting the vibration sensor to an electronic terminal;
the stiffening structure supports two electrical contact outlets, each connected to an electrical terminal, to connect the vibration sensor to an electronic terminal;
the vibration sensor comprises a peripheral seal;
the vibration sensor comprises a protective layer arranged above and at a distance from the stack of layers, the protective layer being rigidly connected to the support layer.

The present disclosure further relates to a non-intrusive device for measuring at least one periodic vital signal of an individual, comprising:
at least one vibration sensor as above, to measure a raw signal representative of the periodic vital signal, and
an electronic terminal connected to the vibration sensor, to analyze and interpret the raw signal and extract the periodic vital signal or an output parameter representative of the periodic vital signal.

The electronic terminal advantageously comprises:
an analog stage for conditioning the raw signal measured by the vibration sensor,
an analog to digital conversion stage of the signal coming from the coming from the conditioning stage,
a digital signal processing stage, for shaping the digital signal and calculating an output parameter representative of the vital signal,
and potentially, a communication stage with an external system.

### BRIEF DESCRIPTION OF THE DRAWINGS

Other features and advantages of the present disclosure will emerge from the following detailed description of embodiments of the present disclosure with reference to the appended figures:

FIGS. 1A and 1B show a vibration sensor according to the present disclosure, respectively, in cross-sectional schematic and perspective views;

FIGS. 2A-2C show a vibration sensor according to the present disclosure, respectively, in schematic cross-sectional, and perspective views;

FIGS. 3A and 3B show a vibration sensor according to the present disclosure, respectively, in cross-sectional schematic and perspective views;

FIGS. 4A-4C show different shapes, in top view, of a vibration sensor according to the present disclosure;

FIGS. 5A-5C show various configurations of devices for measuring a periodic vital signal, according to the present disclosure;

FIG. 6A shows a spectrogram A captured by a vibration sensor according to the present disclosure and a spectrogram Ref picked up by a conventional microphone;

FIG. 6B shows a spectrogram B from the spectrogram A after applying a frequency filter; and a vital signal C, D as a waveform, captured and processed by the device according to the present disclosure.

The same references in the figures may be used for elements of the same type. Some figures contain schematic depictions that, for the sake of readability, are not to scale: In particular, the thicknesses of the layers along the z axis are not to scale with respect to the lateral dimensions along the x and y axes; and the relative thicknesses of the layers between them are not necessarily respected.

The different possibilities (variants and embodiments depicted and/or detailed in the description to follow) must be understood as not being exclusive of one another and may be combined together.

### DETAILED DESCRIPTION

The present disclosure relates to a vibration sensor 100 for measuring at least one periodic, regular or irregular vital signal of an individual. “Individual” is considered here in the broad sense and may correspond to a human being or an animal. The periodic vital signal may be, in particular, the heart rate or the respiratory rate.

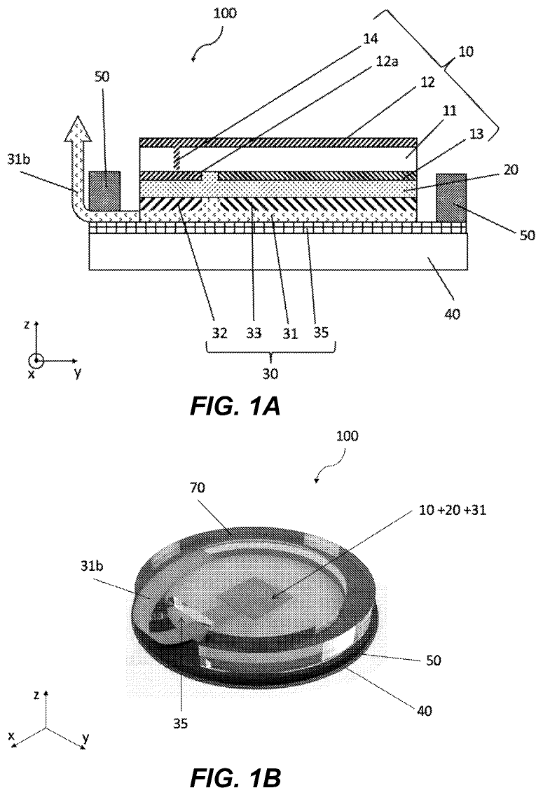

Various configurations of vibration sensors 100 according to the present disclosure are illustrated in FIGS. 1A, 1B, 2A, 2B, 3A and 3B.

The vibration sensor 100 comprises a stack of layers 10 extending parallel to a main plane (x, y), that is to say that the main faces of this stack 10 are substantially parallel to the main plane (x, y) and that the thickness of the stack 10 is measured along an axis z normal to the main plane. The term “layer,” in the present disclosure, implies that the thickness of the layer (or of the stack of layers) is generally significantly less than the lateral dimensions (in the main plane) of the layer.

The stack of layers 10 includes an active layer 11 made of piezoelectric material that has a thickness less than or equal to 20 microns and a Young's modulus greater than or equal to 60 GPa. These physical characteristics give the active layer 11 a high level of sensitivity and to the sensor 100 a high signal-to-noise ratio, for the detection of acoustic waves in the frequencies relating to the targeted periodic vital signals. The low thickness of the active layer 11 also promotes the compactness of the sensor 100.

As is known per se, the active layer 11 made of piezoelectric material will polarize (and therefore generate a flow of charges leading to a measurable electrical signal) if it undergoes a deformation, in particular here, deformation caused by the angular frequency of the periodic vital signal.

Advantageously, the thickness of the active layer 11 is less than or equal to 10 microns, or even less than or equal to 5 microns, to further improve the detection sensitivity of the acoustic waves. It will be ensured that an active layer 11 thickness is sufficient to generate bias voltages typically greater than 500 microvolts during a deformation.

The lateral dimensions (in the main plane (x, y)) of the active layer 11 may be, for example, chosen to be between 500 microns and 50 mm, small dimensions being of course preferred for reasons of compactness of the vibration sensor 100.

The material of the active layer 11 is preferably chosen from piezoelectric ceramics, in a monocrystalline, poly-crystalline or composite form (corresponding to a dispersion of piezoelectric ceramic powder in a matrix, generally polymer). As an example, mention may be made of the following ceramics: lithium niobate (LiNbO₃), lithium tantalate (LiTaO₃), potassium niobate (KNbO₃), (BaTiO₃), quartz (SiO₂), lead magnesium niobate-lead titanate (PMN-PT), lead zirconate titanate (PZT), materials based on potassium sodium niobium lithium antimony (KNN-LS) or modified with calcium titanate (KNN-LS-CT), materials based on potassium sodium lithium niobium tantalum antimony (KNNLNTS), bismuth sodium titanate (BNKLBT), etc.

The stack of layers 10 also includes two contact electrodes 12, 13, arranged on one of the faces of the active layer 11 or on both faces (namely on either side of the active layer 11), to allow free circulation of the charges, set in motion by the polarization (representative of the periodic vital signal) of the layer 11.

Preferably, the contact electrodes 12, 13 have a cumulative thickness less than twice the thickness of the active layer 11, or even less than the thickness of the active layer 11; each electrode 12, 13 therefore advantageously has a thickness of less than 10 microns, or even less than 5 microns.

The contact electrodes 12, 13 may be formed from pure metal materials (for example, Ag, Au, Pd, Pt, Cu, Ni, W or Ti), conductive alloys, or 2D conductive materials (for example, graphene). A diffusion barrier (for example, made of TiN, WN or TaN) and an adhesion layer (for example, made of Cr or Ti) may be provided between the conductive material of each electrode 12.13 and the active layer 11.

Advantageously, the stack of layers 10 consists of the active layer 11 and of the two contact electrodes 12, 13 only.

The vibration sensor 100 also comprises a flexible support layer 30, extending parallel to the main plane (x, y) and including a printed circuit 31 comprising two electrical terminals 32, 33. An electrical connection layer 20 (which also forms part of the vibration sensor 100) is arranged between the stack of layers 10 and the support layer 30, to connect each contact electrode 12, 13 to an electrical terminal 32, 33.

The electrical connection layer 20 is formed by an interposer or by an anisotropic conductive film (ACF). In all cases, the objective is that the two contact electrodes 12, 13 of the stack of layers 10 can be reached at one and the same face of the stack 10; this face (called the lower face) being then associated with the connection layer 20. In the case where the contact electrodes 12, 13 are respectively arranged on the lower face and the other face (called upper face) of the active layer 11, it is advantageous to provide a conductive via 14 passing through the active layer 11 and electrically connecting the electrode 12, arranged on the upper face, to a stud 12a arranged on the lower face and electrically insulated from the other electrode 13 also arranged on the lower face.

An interposer may be composed of thermoplastic (insulating) resin and an electrically conductive material (for example, Nickel) allowing the connection between each contact electrode 12, 13 and an electrical terminal 32, 33.

An anisotropic conductive film (ACF) is conventionally composed of conductive beads dispersed in an insulating polymer matrix; when pressure or thermocompression is applied to the stack of layers 10/ACF 20/support layer 30, vertical electrical conduction is established between electrodes 12a, 13 and terminals 32, 33 (usually in extra thickness) via the conductive beads, whereas the interlayer zones remain insulating.

There are also anisotropic conductive adhesives (ACA) that could be used to form the electrical connection layer 20. These adhesives are based on the same principle as the aforementioned anisotropic conductive film (ACF), with the exception that the polymer matrix is replaced by a liquid precursor capable of being thermally activated to form the final polymer (by polymerization); the final result remains similar to the ACF (conductive beads dispersed in an insulating matrix), but given the fact that the application takes place in the liquid phase, it is possible to drastically reduce the thickness of the electrical connection layer 20.

A more basic solution can also be envisaged, namely the implementation of a conductive paste to connect each electrode and stud of the lower face, to an associated terminal 32, 33, and an insulating filler material to electrically insulate the electrodes 12a, 13 from one another and the terminals 32, 33 from one another.

The electrical connection layer 20 is only in contact with one of the main faces of the stack of layers 10; the edges and the other main face of the stack of layers 10 are totally free, without mechanical contact with the connection layer 20.

The electrical connection layer 20 is therefore at least partially composed of an electrically conductive material and provides a direct vertical connection between electrodes and terminals, conversely to a connection, for example, by cables or wires optionally coated in an insulator. The absence of cables improves the sensitivity of the vibration sensor 100, avoiding the introduction of additional stiffness into the structure, linked to the associated cables and welds.

Preferably, the electrical connection layer 20 is therefore in direct, homogeneous contact against the entire main face of the stack of layers 10. On the side of its other face, the layer 20 is advantageously in direct, homogeneous contact against a face of the support layer 30.

The electrical connection layer 20 typically has a thickness less than 50 microns, in particular, a thickness of between 1 micron and 10 microns.

The support layer 30 is a self-supporting layer, which advantageously has a thickness less than or equal to 500 microns. This gives it the required flexibility.

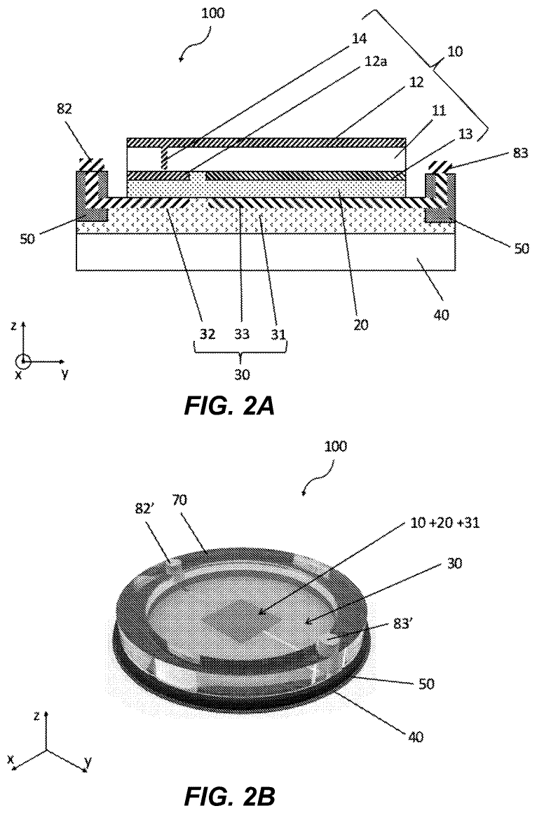

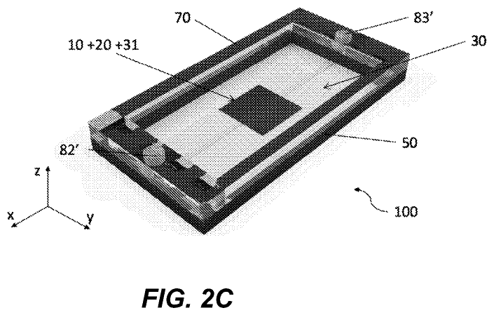

According to one variant, the support layer 30 is essentially composed of the material forming the printed circuit 31 (FIG. 2A): For example, a composite of epoxy resin reinforced with glass fibers. According to another variant, the support layer 30 also comprises a membrane 35, the printed circuit 31 then being situated between the membrane 35 and the electrical connection layer 20 (FIGS. 1A and 3A). The material of the membrane 35, and its thickness, can thus be chosen and adjusted so as to impart the targeted flexibility to the support layer 30. The membrane 35 may be, for example, made of metal, polyvinyl chloride (PVC), or epoxy and glass fibers. By way of example, the membrane 35 (when it is present) may have a thickness of between 50 and 300 microns, and the printed circuit 31 may have a thickness of between 30 and 200 microns.

Typically, the support layer 30 has a stiffness of between 1150000 N/m and 6900000 N/m. The flexible nature of the support layer 30, linked to its thickness and its stiffness, makes it possible to effectively transmit a deformation to the active layer 11, at each pulse of the vital signal.

Advantageously, the stack of layers 10 and the support layer 30, respectively, have a first surface area and a second surface area, in the main plane (x, y), the first surface area being less than or equal to 30% of the second surface. The stack of layers 10 can be arranged in the central part of the support layer 30, in particular, for ease of assembly, or at the periphery to interfere as little as possible with the deformation of the support layer 30, generated by the periodic pulsing of the vital signal that it is sought to measure, the overall objective is to optimize the deformation experienced by the stack of layers 10, as a function of the geometry of the vibration sensor 100.

According to a first embodiment of the sensor according to the present disclosure, the support layer 30 is intended to be in contact with the individual (against their skin or against their clothing or fur): the support layer 30 will then deform due to the periodic pulsing of the vital signal, and transmit this deformation to the active layer 11 of the stack 10.

According to a second embodiment, the vibration sensor 100 further comprises an impedance matching layer 40, which has an acoustic impedance ideally between 5× 10⁵ Pa*s/m and 3×10⁶ Pa*s/m. This acoustic impedance is knowingly chosen close to the acoustic impedance of the muscles and fat (impedance between 1.3×10⁶ and 1.5×10⁶ Pa*s/m), so as to promote the transmission of the pulses of the vital signal to the support layer 30. For example, the impedance matching layer 40 can be formed from silicone (acoustic impedance 1.6×10⁶ Pa*s/m) or of bioplastic, for example, of brand ECOFLEX® (acoustic impedance 1.053×10⁶ Pa*s/m).

The impedance matching layer 40 is arranged against the support layer 30, on a face of the support layer 30 opposite the one in contact with the electrical connection layer 20. The impedance matching layer 40 typically has a thickness greater than or equal to 10 microns, for example, between 50 microns and 5 mm. When the support layer 30 comprises a membrane 35, that membrane is in contact with the impedance matching layer 40.

The impedance matching layer 40 is intended to be in contact with the individual (against their skin or against their clothing or fur). In addition to effectively transmitting the pulses due to its impedance matching with body tissues, this layer 40 also promotes the holding of the sensor 100 against the individual since its flexible and deformable material tends to “adhere” to the contact surface, by adhesion friction. The presence of the impedance matching layer 40, in the second embodiment of the sensor 100, is therefore particularly favorable when the measurement environment is noisy around the individual whose vital signal is to be picked up, and/or when the individual is moving.

In either of the described embodiments, it may be advantageous for the vibration sensor 100 to comprise a peripheral seal 60 surrounding at least the impedance matching layer 40 (when present), as shown in FIGS. 3A and 3B, or surrounding all or part of the support layer 30 (in the absence of an impedance matching layer 40). This seal 60 makes it possible to accommodate the local topology when the sensor 100 is placed in contact with the individual.

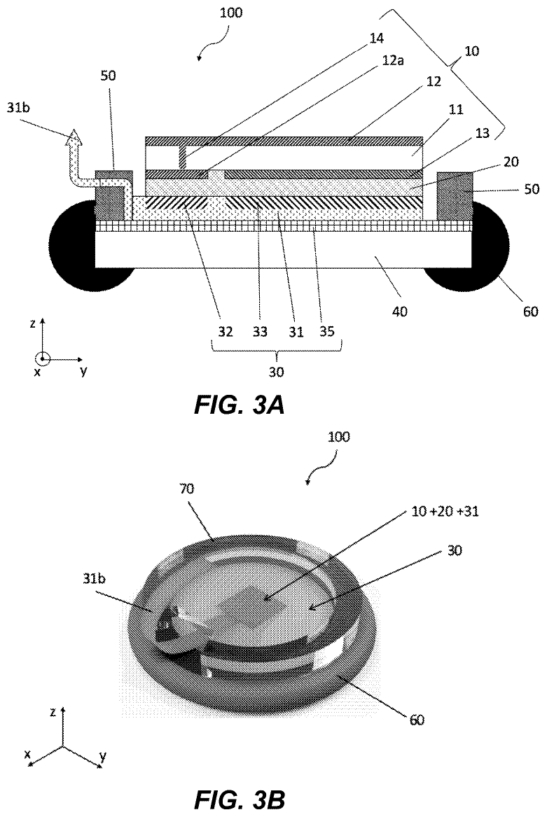

The support layer 30 of the vibration sensor 100 may also comprise a stiffening structure 50, rigidly connected to a peripheral zone of the support layer 30. The function of the stiffening structure 50 is to immobilize the periphery of the support layer 30 and of the impedance matching layer 40 (if present), and thus to accentuate their deformation generated by the periodic pulsing of the vital signal that it is sought to measure. The stiffening structure 50 may take various shapes such as, for example:
a continuous frame (FIG. 4A), advantageously a ring (as shown in FIGS. 1B and 2B), but optionally a rectangle (FIG. 2C), a triangle or another polygon; or
a discontinuous frame, composed of two rigid areas (FIG. 4B), of three rigid areas (FIG. 4C), or even more.

The stiffening structure is advantageously formed from a material having a hardness greater than 30 Shore D, such as PET (polyethylene terephthalate), PMMA (polymethyl methacrylate), PU (polyurethane), PVC (polyvinyl chloride), PP (polypropylene), etc.

Furthermore, given the reduced total thickness of the assembly comprising the stack of layers 10, the connection layer 20, the support layer 30 and potentially the impedance matching layer 40, it may be judicious to provide a system facilitating the handling of the sensor 100 and promoting its robustness: the stiffening structure 50 participates in such a system.

With the aim of further improving the robustness of the vibration sensor 100 and of protecting the active layer 11, in particular, the sensor 100 is preferably provided with a protective layer 70 arranged above and at a distance from the stack of layers 10. The protective layer 70 may advantageously be rigidly connected to the stiffening structure 50. As shown in FIGS. 1B, 2B, 2C and 3B, this protective layer 70 may comprise, in particular, a plastic shell, for example, 500 microns thick. Because it is located at a distance (along the z axis in the figures) from the stack of layers 10 (and thus without contact with the stack), it does not disturb the deformation thereof in connection with the support layer 30.

Note that FIGS. 1B, 2B and 3B illustrate vibration sensors 100 of a generally circular shape, in the main plane (x, y), comprising a stack of layers 10 of square shape. Any other form, both for the stack of layers 10 and for the support layer 30 (and for the other layers of the assembly forming the sensor 100), is of course conceivable.

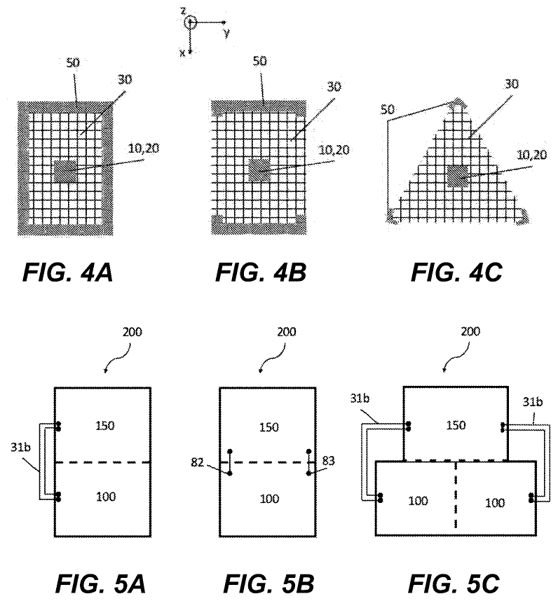

The vibration sensor 100 according to the present disclosure is defined to form part of a non-intrusive device 200, for measuring at least one periodic vital signal of an individual. Such a device 200, which is also a subject matter of the present disclosure, comprises at least one vibration sensor 100 as described above, to measure a raw signal (linked to the characteristic periodic vital signal of the individual), and an electronic terminal 150 connected to the vibration sensor 100, to analyze and interpret the raw signal and then extract the periodic vital signal or information relating to this vital signal. The device 200 may comprise a vibration sensor 100 (FIGS. 5A and 5B) or a plurality (two, or even more) of sensors 100 connected to the electronic terminal 150 (FIG. 5C). When there are a plurality of sensors 100, it is possible to measure the same signal or different vital signals (heart rate and breathing), from the same individual or of several individuals (such as, for example, a pregnant woman and her baby).

As mentioned above, the support layer 30 or, when it is present, the impedance matching layer 40 of the vibration sensor 100 is intended to be arranged against the individual, either directly against the skin, or against clothing (human being), fur (animal), or the like. The sensitivity of the vibration sensor 100 makes it possible to capture a raw signal, regardless of the configuration (skin contact or intermediate layer like clothing).

The vibration sensor 100 according to the present disclosure further has the benefit of greatly attenuating the frequencies located outside the range of frequencies of interest (range of frequencies typically between 0.2 Hz and 500 Hz for heart and respiratory rates, or even frequencies less than or equal to 70 Hz): it has been observed, in particular, that speech and other environmental sounds do not contaminate the measured signal, most particularly in the case of the second embodiment involving the impedance matching layer 40. The sound environment of the individual at the time the measurement is taken therefore does not need to be calm and silent; the individual does not need to remain immobile. This greatly expands the possibilities for tracking vital signals, and does so under less restrictive conditions than with the devices of the prior art.

It will preferably be chosen to place the vibration sensor 100 (i.e., the support layer 30 or optionally the impedance matching layer 40) against a zone of the body at which the vital pulse (respiratory rate, heart rate) that it is desired to measure is palpable to the touch.

To connect the vibration sensor 100 and the electronic terminal 150, the printed circuit 31 of the sensor 100 may comprise a wire connection element 31b, for example, a strip in the form of a sheet, as shown in FIGS. 1A, 1B and 3A, 3B. The end piece of the wire connection element 31b comprises electrical contact connectors, connected to the electrical terminals 32, 33 of the printed circuit 31, which can be connected to the electronic terminal 150. The electronic terminal 150 may, in this case, be located at a distance from the sensor 100, in particular, on a module for fastening to the individual (for example, a pocket, a belt, a bracelet, etc.). Still remote from the sensor 100, the electronic terminal can be connected or integrated to a more complex external system, such as a monitor that is fixed or optionally transportable.

Alternatively, the stiffening structure 50 of the vibration sensor 100 may be the support of two electrical contact outlets 82, 83, each connected to an electrical terminal 32, 33 of the printed circuit 31, as can be seen in FIG. 2A. In such a case, the electronic terminal 150 can be directly superimposed on the stiffening structure 50, and electrically connected to the sensor 100 via the two electrical contact outlets 82, 83. According to one variant, the protective layer 70 may provide intermediate contact plugs 82′, 83′, relaying the electrical contact outlets 82, 83 and intended to be connected to the electronic terminal 150 (FIGS. 2B and 2C); the latter can then be directly arranged on the protective layer 70. In these configurations, where the terminal 150 is superimposed on the sensor 100, the device 200 can take a particularly compact form and form a portable and potentially autonomous device.

The terminal 150 comprises various electronic stages enabling it to analyze and interpret the raw signal measured by the vibration sensor 100. An analog stage for conditioning the raw signal measured by the vibration sensor 100 will first amplify and filter the electrical signal received from the sensor 100. This stage is typically composed of a first block of the charge amplification type whose resistance ratio sets the amplification gain of the electrical signal received from the sensor 100, and a second block of the Sallen & Key filter type making it possible to filter the frequencies beyond the acoustic spectrum of the targeted vital signals. The electronic terminal 150 then comprises a stage of analog to digital conversion of the signal coming from the conditioning stage. Then, a processing stage of the digital signal, composed of a microcontroller, performs the shaping of the signal by calculating a Shannon energy envelope function. Finally, from the shaped signal, the output parameter of interest, representative of the vital signal, can be calculated.

The collected data, relating to the vital signal or the output parameter of interest, can be stored for subsequent analysis, or be interpreted in real time and trigger the response of a secondary system comprised in the device 200 or an external one. The response could be an information feedback (visual, acoustic, mechanical, etc.) and/or the triggering of one or more actions, for example:
mechanical(s): opening/closing of a system,
electrical(s): turning on/turning off/varying a system, hydraulic, pneumatic, thermal, etc.

To authorize the transmission of the output parameter of interest to a possible external system, the electronic terminal 150 may comprise a communication stage. Known connection protocols (CAN, UART, USB) or wireless data transmission, (Wi-Fi, Bluetooth, etc.) may be used, for example.

In the case of a portable device 200, a battery, preferentially rechargeable, can be provided to supply power to the various aforementioned stages of the electronic terminal 150.

The vibration sensor 100 and the non-intrusive device 200 for measuring a periodic vital signal according to the present disclosure can address a number of fields of application, in the fields of medicine, health, transportation, industry, sports, or leisure.

As mentioned above, the device 200 can be broken down into various configurations:
a portable and autonomous device, in a compact form (superimposed sensor 100 and terminal 150) or in a dissociated form (sensor 100 connected to the terminal 150 by a wire connection element 31b);
a portable device, in compact form or in a dissociated form, wherein the terminal 150 is configured to be wired to a more complex external system (in particular, a monitor), for example, in a backup vehicle or in a medical examination room;
a fixed device, in a compact form or in a dissociated form, wherein the terminal 150 is connected by wire or integrated with a fixed and more complex external system.

### Example Embodiment

An example of manufacturing the vibration sensor 100 and the device 200 will now be described. Of course, this example is not limiting because there are other methods for stacking and assembling different types of layers, capable of being implemented to produce the sensors 100 and device 200 that are the subject matter of the present disclosure.

In order to manufacture the stack of layers 10 of the vibration sensor 100, it is, in particular, possible to use a transfer method close to that described by T Dufay et al. in the publication “Flexible PZT thin film transferred on polymer substrate” (Surface and Coatings Technology, Elsevier, 2018, 343, pp. 148-152).

A solution of PZT precursor is deposited by spin-coating on a sacrificial substrate (for example, aluminum), to form a viscous layer. An opening is made through the layer in order to allow the passage of an electrical path. Then, a heat treatment at 650° C. is applied to crystallize the PZT and form an active layer 11 made of piezoelectric material with a thickness of 5 microns.

A platinum contact electrode 12, of 400 nm thickness, is deposited by a chemical vapor deposition technique (for example, PECVD) on the upper (free) face of the active layer 11 made of PZT, then covered with a polyurethane adhesive layer. An opening is also made through the electrode/adhesive layer stack for the passage of the electrical path. A temporary layer made of polymer (for example, PET), 200 microns thick, is attached to the thermal compression polyurethane adhesive layer, to facilitate the handling of the active layer 11. The temporary layer is open to allow the passage of the electrical path, and filled with conductive glue, which will form the conductive via 14, in electrical contact with the contact electrode 12. The sacrificial substrate is then chemically etched until the lower face of the active layer 11 made of PZT is bare. The other contact electrode 13 and the stud 12a, in electrical contact with the via 14, are formed by aluminum deposition (about 400 nm) on the lower face of the PZT.

This manufacturing method can allow the creation of a PZT film having large lateral dimensions, which are then cut to define the active layer 11 with the lateral dimensions desired for its integration into the vibration sensor 100 according to the present disclosure. In the example described, the active layer 11 has lateral dimensions (along the main plane (x, y)) of 5 mm by 15 mm.

A printed circuit board (PCB) 31 is then chosen having a thickness of 100 microns, lateral dimensions substantially identical to those of the active layer 11 and comprising two electrical terminals 32, 33. An anisotropic conductive film (ACF) 20 is laminated on the printed circuit 31. Using a handling machine (of the “Pick and Place” type), the active layer 11 is positioned opposite the connection layer 20, so that each electrode 12a, 13 (on the lower face of the active layer 11) is in line with an electrical terminal 32, 33 of the printed circuit 31; then an assembly by thermocompression is carried out.

The temporary polymer layer can then be removed.

The printed circuit 31 is then bonded to a PVC membrane 35, with a thickness of 300 microns and lateral dimensions 50 mm, to finalize the formation of the support layer 30.

An impedance matching layer 40 made of silicone, of thickness 3 mm, can be assembled by lamination, screen printing or molding against the membrane 35.

A polypropylene stiffening structure 50 and a silicone peripheral seal 60 are attached to the periphery of the membrane 35 by fitting. A polypropylene cover, forming the protective layer 70 above and at a distance from the active layer 11, is poured, injected or laminated onto the stiffening structure 50.

In this example, the printed circuit 31 comprises a wire element 31b (web) that makes it possible to connect the electrical terminals 32, 33 of the printed circuit 31 to the electronic terminal 150, via electrical contact plugs. The terminal 150 comprises the electronic stages set out in the general description.

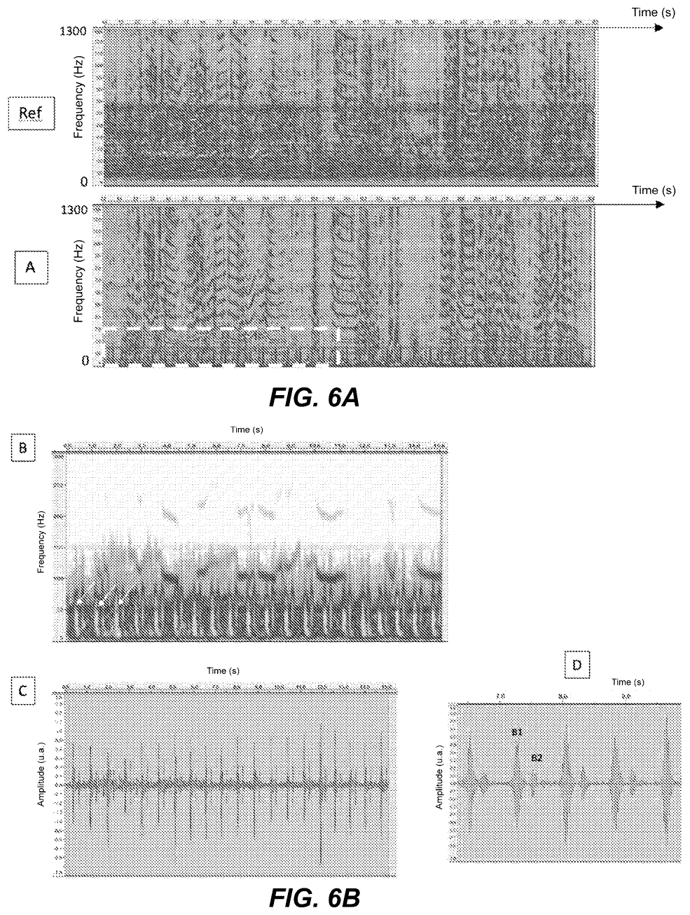

With the device 200 thus formed, an example of application to the measurement of the heart rate of a human being is shown in FIGS. 6A and 6B.

The cardiac cycle comprises two phases: the first is a contraction phase (systole) and the second is a relaxation phase (diastole). During systole, blood is ejected from the chambers of the heart, and during diastole, the chambers are filled with blood. Ventricular systole leads to the closure of the mitral and tricuspid valves. The heart sounds are named based on their place in the cardiac cycle and occur at specific points thereof. The initial cardiac sound is called the first heart sound S1. It occurs at the beginning of ventricular systole when the ventricular volume is maximum. The first sound S1 corresponds to a point that appears early in the elevation of the ventricular pressure curve, when the latter becomes greater than the atrial pressure and the mitral and tricuspid valves close. This corresponds to the QRS complex of the ECG (electrocardiogram). On a graphic recording of heart sounds, called phonocardiogram, this is the first of the recorded components. The second heart sound S2 occurs at the end of the ventricular systole, at the time of the dicrotic wave on the ventricular pressure curve, when the pulmonary and aortic valves close. This is the second of the components recorded on a phonocardiogram. The period between S1 and S2 represents ventricular systole.

The cardiac acoustic spectrum typically extends between 0 and 1300 Hz. However, most of the acoustic power emitted by the heart is below 70 Hz.

Recall that the level of the “speaking” voice differs by sex and age, but is typically between 75 Hz and 450 Hz. This makes it possible to separate cardiac and voice information.

For the measurement of the heart rate, the device 200 is placed on the thorax of the individual, substantially on the left, the impedance matching layer 40 being placed in contact with their clothing (in this example, two thicknesses of cotton and wool clothing).

FIG. 6A shows a raw spectrogram A, acquired on a frequency scale ranging from 0 to 1300 Hz, by the vibration sensor 100 according to the present disclosure (acquisition frequency 128 kHz). The spectrogram Ref corresponds to the acquisition (in parallel with the measurement made by the vibration sensor 100 of the heart rate) by a conventional microphone, of the signal relating to the sound environment: The individual for whom the measurement of the vital signal is carried out is speaking with other people, the sound environment is therefore noisy, as can be seen from the reference spectrogram Ref.

In FIG. 6B, an extract B of 15s of the spectrogram A is reported, on a frequency scale 0-300 Hz, after applying a low-pass filter at 300 Hz. It will be noted that regular peaks (pointed by the white arrows on the spectrogram B), in the 0-70 Hz frequency range, are very clearly identifiable, they correspond to the heart rate measured by the vibration sensor 100 according to the present disclosure. By cutting at 300 Hz, the majority of the information relating to the cardiac pulses is preserved but a lot of parasitic frequencies are eliminated, in particular, related to speech and to any other surrounding noise between 300 and 1300 Hz that may be experienced particularly in an aerial, nautical, or motor vehicle (individual, collective, emergency, or other), or in a noisy place in general.

The peaks indicated on the spectrogram B can be visualized in the form of a wave: This is the signal C, shown in FIG. 6B. A zoom-in D on this signal C reveals the peaks representative of the first noise S1 and of the second noise S2 corresponding to the heart rate of the individual.

From the signals C and D, it is possible to extract the periodic signal and/or an output parameter, representative of the heart rate of the individual.

As has just been illustrated and generally, the vibration sensor 100 and the non-intrusive device 200 for measuring a periodic vital signal according to the present disclosure provide reliable information regarding the vital signal, regardless of the sound environment and the activity of the individual at the time of measurement; they further relax the measurement constraints, since they do not require direct contact with the skin of the individual.

Of course, the present disclosure is not limited to the described embodiments and examples, and variant embodiments can be provided thereto without departing from the scope of the invention as defined by the claims.

1. A vibration sensor for measuring at least one periodic vital signal of an individual, the vibration sensor comprising:
a stack of layers extending parallel to a main plane and including an active layer of piezoelectric material and two contact electrodes arranged on at least one face of the active layer, the active layer having a thickness less than or equal to 20 microns and a Young's modulus greater than or equal to 60 GPa;
a flexible support layer configured to transmit a deformation to the active layer of the stack of layers at each pulse of the vital signal, the support layer extending parallel to the main plane and including a printed circuit comprising two electrical terminals; and
an electrical connection layer, arranged between the stack of layers and the support layer, to connect each contact electrode to an electrical terminal,
wherein the vibration sensor is configured to be placed in contact with the individual, on the side of the support layer.

2. The vibration sensor of claim 1, further comprising an impedance matching layer having an acoustic impedance between 5×10⁵ Pa*s/m and 3×10⁶ Pa*s/m, and arranged on a face of the support layer opposite the face of the support layer in contact with the electrical connection layer.

3. The vibration sensor of claim 1, wherein the piezoelectric material of the active layer comprises a ceramic material in monocrystalline, poly-crystalline or composite form.

4. The vibration sensor of claim 1, wherein:
the contact electrodes have a cumulative thickness of less than twice the thickness of the active layer;
the support layer is self-supporting and has a thickness of less than or equal to 500 microns; and
the impedance matching layer has a thickness greater than or equal to 10 microns.

5. The vibration sensor of claim 1, wherein the electrical connection layer comprises an interposer or an anisotropic conductive film.

6. The vibration sensor of claim 1, wherein the support layer includes a membrane arranged on a face of the printed circuit opposite the face of the printed circuit in contact with the electrical connection layer.

7. The vibration sensor of claim 1, wherein the stack of layers and the support layer, respectively, have a first surface area and a second surface area, in the main plane, the first surface area being less than or equal to 30% of the second surface area.

8. The vibration sensor of claim 1, wherein the support layer comprises a stiffening structure rigidly connected to a peripheral zone of the support layer.

9. The vibration sensor of claim 1, wherein the printed circuit comprises a wire connection element connecting the vibration sensor to an electronic terminal.

10. The vibration sensor of claim 8, wherein the stiffening structure supports two electrical contact outlets each connected to an electrical terminal, to connect the vibration sensor to an electronic terminal.

11. The vibration sensor of claim 1, further comprising a peripheral seal.

12. The vibration sensor of claim 1, further comprising a protective layer arranged above and at a distance from the stack of layers, the protective layer being rigidly connected to the support layer.

13. A non-intrusive device for measuring at least one periodic vital signal of an individual, the non-intrusive device comprising:
at least one vibration sensor according to claim 1 for measuring a raw signal representative of the periodic vital signal, and
an electronic terminal connected to the vibration sensor for analyzing and interpreting the raw signal and extract the periodic vital signal or an output parameter representative of the periodic vital signal.

14. The device of claim 13, wherein the electronic terminal comprises:
an analog stage for conditioning the raw signal measured by the vibration sensor;
an analog to digital conversion stage of the signal coming from the conditioning stage; and
a digital signal processing stage for shaping the digital signal and calculating an output parameter representative of the vital signal.

15. The device of claim 13, wherein the electronic terminal is configured to communicate with an external system.

16. The device of claim 13, wherein the at least one vibration sensor further comprises an impedance matching layer having an acoustic impedance between 5×10⁵ Pa*s/m and 3×10⁶ Pa*s/m, and arranged on a face of the support layer opposite the face of the support layer in contact with the electrical connection layer.

17. The device of claim 13, wherein the piezoelectric material of the active layer of the at least one vibration sensor comprises a ceramic material in monocrystalline, poly-crystalline or composite form.

18. The device of claim 13, wherein:
the contact electrodes of the at least one vibration sensor have a cumulative thickness of less than twice the thickness of the active layer;
the support layer of the at least one vibration sensor is self-supporting and has a thickness of less than or equal to 500 microns; and
the impedance matching layer of the at least one vibration sensor has a thickness greater than or equal to 10 microns.

19. The device of claim 13, wherein the electrical connection layer of the at least one vibration sensor comprises an interposer or an anisotropic conductive film.

20. The device of claim 13, wherein the support layer of the at least one vibration sensor includes a membrane arranged on a face of the printed circuit opposite the face of the printed circuit in contact with the electrical connection layer.
